SEQUENCE LISTING (8 sequences)

SEQ ID NO: 1 - DNA, 22 bp, Artificial Sequence
  Adiponectin forward primer
aaggagatccaggtcttattgg

SEQ ID NO: 2 - DNA, 18 bp, Artificial Sequence
  Adiponectin reverse primer
accttcagccccgggtac

SEQ ID NO: 3 - DNA, 20 bp, Artificial Sequence
  PPAR-gamma forward primer
cataaagtccttcccgctga

SEQ ID NO: 4 - DNA, 20 bp, Artificial Sequence
  PPAR-gamma reverse primer
gggggtgatgtgtttgaact

SEQ ID NO: 5 - DNA, 21 bp, Artificial Sequence
  IL-6 forward primer
ggtacatcctcgacggcatct

SEQ ID NO: 6 - DNA, 21 bp, Artificial Sequence
  IL-6 reverse primer
gtgcctctttgctgctttcac

SEQ ID NO: 7 - DNA, 20 bp, Artificial Sequence
  Insulin receptor forward primer
tgcaaacccaagaacgtcag

SEQ ID NO: 8 - DNA, 20 bp, Artificial Sequence
  Insulin receptor reverse primer
agcttccgggagttcagtac

(19) United States
(12) Patent Application Publication
(10) Pub. No.: US 2022/0112464 A1
(43) Pub. Date: Apr. 14, 2022

(54) VASCULARIZED ORGANOID MODEL INCORPORATING ISOLATED HUMAN MICROVESSEL FRAGMENTS

(71) Applicant: Advanced Solutions Life Sciences, LLC, Louisville, KY (US)

(72) Inventors: Hannah A. Strobel, Manchester, NH (US); James B. Hoying, Manchester, NH (US)

(21) Appl. No.: 17/497,364

(22) Filed: Oct. 8, 2021

Related U.S. Application Data
(60) Provisional application No. 63/089,907, filed on Oct. 9, 2020.

(51) Int. Cl. C12N 5/077; G01N 33/50

(52) U.S. Cl. CPC C12N 5/0653; G01N 33/5082; G01N 33/5014; C12N 2501/33; C12N 2501/165; C12N 2501/999; C12N 2506/1353

(57) ABSTRACT

A method for producing a functional, vascularized organoid or spheroid is provided, the method including: (a) mixing a suspension of stromal cells with microvessel (MV) fragments isolated from adipose tissue to provide an MV/stromal cell suspension; and (b) culturing the MV/stromal cell suspension in an angiogenic medium to provide the functional, vascularized organoid or spheroid. Also provided is a method for producing a functional, vascularized adipocyte organoid or spheroid and a method of screening compounds for pharmacological or toxicological activity, using the vascularized organoids and/or spheroids provided herein.

### CROSS REFERENCE TO RELATED APPLICATION

This application claims priority to U.S. Provisional Application Ser. No. 63/089,907, filed Oct. 9, 2020, the entire contents of which are incorporated herein by reference.

### STATEMENT REGARDING FEDERALLY SPONSORED RESEARCH OR DEVELOPMENT

This invention was made with Government support under 1842675 awarded by National Science Foundation. The Government has certain rights in the invention.

### TECHNICAL FIELD

This disclosure relates to the field of organoids and spheroids. Specifically, this disclosure relates to functional, vascularized organoids and spheroids comprising microvessel fragments, and methods of producing the same.

### SEQUENCE LISTING

A computer-readable form (CRF) sequence listing having file name Sequence_Listing_AVN0081PA.txt, created on Oct. 6, 2021, is incorporated herein by reference. The nucleic and amino acid sequences listed in the accompanying sequence listing are shown using standard abbreviations as defined in 37 C.F.R. 1.822.

### BACKGROUND

Tissue organoids and spheroids are currently in use to study cellular behavior, interrogate tissue biology dynamics, and develop new pharmaceuticals. In some cases, organoids may also serve as building blocks for larger engineered tissues to be implanted. As the utility of organoids expands, the composition of and approaches to building organoids have also progressed. Importantly, the inclusion of vascular cells, typically endothelial cells (ECs), into the organoid is seen as necessary to recapitulate more of the relevant native tissue biology, and potentially provide a precursor to engraftment and perfusion. In addition to endothelial cells, other cell types, such as smooth muscle cells, macrophages, stem cells, pericytes, and other immune cells can comprise and/or associate with the microvessel wall and coordinate with ECs to influence angiogenesis, network formation, and vascular function. These vascular cells also interact with other cellular components within the tissue to establish homeostasis, function, and, when dysregulated, disease.

Efforts to vascularize organoids in vitro have primarily focused on incorporating ECs as precursors to forming vessel segments. In these cases, ECs self-assemble into a small number of capillary-like structures within organoids. While capturing some aspects of the vasculature, these single-EC type structures lack the structural and cellular complexity of the native microvasculature. Reflecting this, more complex vascular elements within organoids have been produced by incorporating multiple microvascular cell types derived from induced pluripotent stem cells. This, however, requires lengthy and complicated differentiation procedures. There is still a strong need for an organoid vascularization solution that is robust, simple to implement, and translatable to multiple tissue systems.

### SUMMARY

Provided herein are functional, vascularized organoids and/or spheroids comprising living microvessel fragments capable of inosculation, thereby providing an organoid model that more closely approximates an organ or tissue microenvironment.

In one embodiment, a method for producing a functional, vascularized organoid is provided, the method comprising: (a) mixing a suspension of stromal cells with microvessel (MV) fragments isolated from adipose tissue to provide an MV/stromal cell suspension; and (b) culturing the MV/stromal cell suspension in an angiogenic medium to provide the functional, vascularized organoid.

In another embodiment, a method for producing a functional, vascularized adipocyte organoid is provided, the method comprising: (a) culturing mesenchymal stem cells (MSCs) in an adipocyte differentiation medium (ADM) to provide committed pre-adipocyte cells; (b) mixing a suspension of committed pre-adipocyte cells with microvessel (MV) fragments isolated from adipose tissue to provide an MV/pre-adipocyte suspension; and (c) culturing the MV/pre-adipocyte suspension in an adipocyte maintenance medium (AMM) to provide the functional, vascularized adipocyte organoid.

In another embodiment, a method of screening a compound for pharmacological or toxicological activity is provided, the method comprising: (a) providing a vascularized organoid or spheroid comprising stromal cells and isolated microvessel (MV) fragments; (b) administering a test compound to the organoid or spheroid; and (c) detecting a pharmacological or toxicological response of the organoid or spheroid.

These and other objects, features, embodiments, and advantages will become apparent to those of ordinary skill in the art from a reading of the following detailed description and the appended claims.

### BRIEF DESCRIPTION OF THE DRAWINGS

The details of embodiments of the presently-disclosed subject matter are set forth in this document. Modifications to embodiments described in this document, and other embodiments, will be evident to those of ordinary skill in the art after a study of the information provided in this document.

FIGS. 1A-1E demonstrate that mesenchymal stem cells (MSCs) stimulate angiogenesis. Microvessels were co-cultured with MSCs in transwell inserts (FIG. 1A, FIG. 1B), or without MSCs (FIG. 1C, FIG. 1D). Culture mediums either contained no VEGF (FIG. 1A, FIG. 1C), or 50 ng/ml VEGF (FIG. 1B, FIG. 1D). Wells with lectin stained microvessels were scanned with a confocal microscope and processed with BioSegment software to quantify microvessel length density in each well. Average vessel densities for each group are shown in (FIG. 1E) after 6 days of culture. N=3, bars are mean±SD. *P<0.05 compared to all other groups, one-way ANOVA with Nueman-Keuls post hoc test.

FIGS. 2A-2H characterize vascularized MSC organoids. MSCs and microvessels (MVs) were mixed and seeded at MV:MSC ratios of 1:12.5 (FIG. 2A), 1:25 (FIG. 2B), 1:50 (FIG. 2C), or 1:100 (FIG. 2D). The number of sprouts growing out of each organoid into the surrounding collagen matrix was counted and normalized to the circumference of the organoid (FIG. 2E, FIG. 2F). Images of (FIGS. 2A-2D) were taken after a total of 7 days of culture (5 days as organoids, 2 days as organoids embedded in collagen). An earlier time point is shown in (FIG. 2G, FIG. 2H), where organoids are embedded at day 2 and fixed and stained at day 4 (1:50 group shown). Red=lectin (endothelial cells), blue=Hoechst (nuclei), scale=200 μm. One way ANOVA with Neuman-Keuls post hoc test. *P<0.05 compared to both 1:12.5 and 1:100, N=3, bars are mean±SD.

FIGS. 3A-3F demonstrate the effects of collagen incorporation on organoid vascularization. Organoids were seeded without collagen (FIG. 3A, FIG. 3B), or with collagen (FIG. 3C, FIG. 3D), in the seeded cell suspension. Vascularization can be clearly seen in confocal images of embedded organoids (FIG. 3A, FIG. 3C; lectin stain, red=endothelial cells). A picrosirius red/fast green stain shows collagen remaining in MSC organoids after 7 days of culture (FIG. 3B, 3D; red=collagen, green=counterstain). The number of angiogenic sprouts growing out the organoids were not significantly different between the two groups (FIG. 3E), although organoids with collagen maintained a larger diameter (FIG. 3F). Student's t-test, *P<0.05, bars are mean±SD, N=6-7.

FIGS. 4A-4G characterize 2D cell cultures after differentiation. Oil Red 0 staining with a hematoxylin counterstain shows lipid droplets (lipids=red, nuclei=purple) in cells treated with ADM for 24 days (ADM; FIG. 4A), treated with ADM for 17 days, then AMM for 7 days (AMM; FIG. 4B), or were cultured in 10% FBS (FBS; FIG. 4C). A lipolysis assay shows glycerol production in response to isoproterenol in each of the three treatments (FIG. 4D). PCR gel shows expression of adiponectin, PPAR-γ, and GAPDH FIG. 4E). RT-PCR shows comparable expression of both adiponectin (FIG. 4F) and PPAR-γ (FIG. 4G) in both ADM and AMM groups, which are compared to FBS. *P<0.05 compared to all other groups, One-way ANOVA with Neuman-Keuls post-hoc analysis. N=3. Bars are mean±SD.

FIGS. 5A-5E depict vascularized adipose-like organoids with collagen inclusion. H&E stain of organoids seeded with collagen and pre-adipocytes, without microvessels (FIG. 5A), or with microvessels (FIG. 5B), after 7 days of culture in AMM. Fluorescent BODIPY stained image shows apparently mature adipocytes within MV containing organoid, characterized by large round lipid droplets (FIG. 5C). After 2 days of embedded culture (7 days total organoid culture), microvessels can be seen growing throughout the organoid (FIG. 5D, FIG. 5E; lectin stain, red=endothelial cells). FIG. 5E is a high magnification inset of FIG. 5D.

FIGS. 6A-6D depict vascularized adipose-like organoids without collagen inclusion. H&E stain of organoids seeded with pre-adipocytes but no included collagen, either without microvessels (FIG. 6A), or with microvessels (FIG. 6B), after 7 days of culture in AMM. After 2 days of embedded culture (7 days total organoid culture), some neovessel sprouts can be seen growing out of the organoid into the surrounding matrix (FIG. 6C, FIG. 6D; lectin stain, red=ECs). FIG. 6D is high magnification inset of FIG. 6C.

FIGS. 7A-7D relate to adipose organoid function. Lipolysis assay showing glycerol production in organoids in response to isoproterenol treatment (FIG. 7A). PCR shows expression is PPAR-γ, adiponectin, and GAPDH in both groups (FIG. 7B). RT-PCR shows relative expression of adiponectin (FIG. 7C) and PPAR-γ (FIG. 7D) in organoids with microvessels compared to organoids with no microvessels. P>0.05, student's t-test.

FIGS. 8A-8C demonstrate microvessel effect on insulin receptor expression. RT-PCR indicated MVs increase insulin receptor expression compared to organoids without MVs (FIG. 8A). Immunohistochemistry shows receptor expression in organoids without (FIG. 8B) and with (FIG. 8C) MVs. Green=insulin receptor, red=endothelial cells (lectin), blue=nuclei (Hoechst); scale=50 *P<0.05, student's t test.

FIGS. 9A-9D depict results of TNF-α challenge on organoids. Organoids with and without MVs were treated with or without TNF-α for 24 hours. An ELISA was used to measure secretion of the inflammatory cytokine IL-6 (FIG. 9A). PCR shows the effect of TNF-α on IL-6 gene expression (FIG. 9B) and the adipocyte markers adiponectin and PPAR-γ (FIG. 9C, FIG. 9D). *P<0.05 compared to all other groups. **P<0.05 compared to groups with TNF-α treatment. One-Way ANOVA on Ranks with Neuman-Keuls post hoc analysis (FIG. 9A), or One-Way ANOVA with Neuman-Keuls post hoc analysis (FIG. 9B, FIG. 9C, FIG. 9D).

FIGS. 10A-10D are images depicting organoids according of present disclosure including MSCs (FIGS. 10A-10C) and excluding MSCs but including VEGF (FIG. 10D).

FIG. 11 is a dot plot showing organoid diameter (mm) vs. vascularization (sprouts/circumference).

FIG. 12 is an image depicting the cross section of a vascularized organoid, wherein the arrows indicate vessel lumens.

FIG. 13 is a graph depicting vessel length (mm) of organoids cultured under a variety of conditions (serum free+VEGF, 10% FBS, RPMI/B-27/VEGF, ADM, AMM, RPMI/B-27+IM, RMPI/B-27+Dex, RPMI/B-27+IBMX).

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an” and “the” include plural references unless the content clearly dictates otherwise.

Spheroids are 3D spherical cell aggregates that are developed via 3D cell culture techniques. Spheroids are generally understood to be less advanced compared to organoids, and may lack the higher organization characteristic of organoids.

Organoids are complex, 3D, multicellular aggregates of organ-specific cells developed from stem cells or progenitor cells, which self-organize in a manner similar to an in vivo organ using 3D cell culture techniques. Organoids comprise multiple organ-specific cell types that are grouped together and spatially organized in a manner similar to the organ, and are capable of recapitulating specific function(s) of the organ. Organoids approximate the in vivo microenvironment of the organ and are useful in research, as models of disease, and in directing personalized medicine. Organoids may be cultured from allogeneic or autologous stem or progenitor cells.

Stromal cells are a heterogeneous class of cells that play a role during development, tissue injury, regeneration, immune response, cancer, and other pathologies. Stromal cells are differentiating cells that can become connective tissue cells of any organ, such as the uterine mucosa, prostate, bone marrow, lymph node, and ovary. Stromal cells are stored in the bone marrow and throughout other tissues of the body. Stromal cells may be found in adipose tissue, endometrium, synovial fluid, dental tissue, amniotic membrane and fluid, and placenta.

Mesenchymal stem cells (MSCs) are stromal cells derived from various sources, such as bone marrow or adipose tissue. MSCs are native cells that may differentiate to a variety of cell types, including osteoblasts, osteocytes, chondrocytes, myocytes, and adipocytes (fat cells that give rise to marrow adipose tissue).

Isolated microvessel fragments harvested from adipose tissue have proven effective at deriving new microvasculatures in a variety of applications. Microvessels are derived from all 3 general microvascular compartments (i.e. arterioles, venules, and capillaries), retain their intact native structure (including lumen) and cellular composition, and readily recapitulate angiogenesis and tissue vascularization when placed in 3D environments. This is true for microvessels derived from mouse, rat, and human. Importantly, as neovessels sprout and grow from the parent microvessels, they locate and inosculate with each other creating a network of neovessels that fills the tissue space. Additionally, the microvasculatures derived from the isolated microvessels is adaptive, capable of acquiring an organotypic phenotype in the presence of tissue-specific parenchyma and stromal cells. When implanted, microvessels rapidly inosculate with host vasculature to perfuse the implanted region. Recently, it has been shown that stromal cells are important in guiding neovessels across tissue boundaries such as that present between a graft and the implant tissue.

Isolated microvessel fragments are combined with mesenchymal stem cells (MSCs) according to the methods disclosed herein to provide functional, vascularized organoids or spheroids. Accordingly, a protocol for co-seeding microvessels with MSCs in a self-assembled organoid format is provided. MSCs are advantageously employed in the present models, as they can be easily harvested and are conducive to creating patient-specific tissues and disease models. MSCs can be differentiated into a variety of tissue types, including adipose, bone, smooth muscle, and cartilage.

As disclosed herein, to demonstrate the versatility of the disclosed vascularization system and show its effectiveness in other tissues, microvessels were incorporated into adipose-like organoids. Adipose plays a key role in several metabolic diseases, including diabetes and obesity. Better understanding the complex changes in function and signaling that occur in diseased states may accelerate development and testing of new treatments and therapies. While efforts have been made to develop adipose spheroids for this purpose, none has achieved the vascular complexity found in vivo. This disclosure demonstrates that microvessels can be used to fabricate functional, vascularized, adipose-like organoids for use in adipose tissue modeling.

The integration of microvessels with adipocyte precursors requires a staged approach to facilitate both adipocyte differentiation and angiogenesis, since the media commonly used to differentiate stem cells into adipocytes in fact inhibits angiogenesis.

The disclosed organoid fabrication protocol, with or without microvessels, produces differentiated adipocytes while enabling the addition of other elements, such as stromal matrix. This strategy accommodates the use of primary cell sources, MSCs, and human microvessels to capture more accurate biology and enable personalized medicine-related uses. The presently disclosed methods employ MSC and microvessel donor lots with comparable functional outcomes. Despite the challenges of donor variation, heterogeneity can be leveraged to pursue new insights into the variations of adipose biology in the healthy and diseased populations.

### Methods for Producing Organoids

In one embodiment, a method for producing a functional, vascularized organoid is provided, the method comprising: (a) mixing a suspension of stromal cells with microvessel (MV) fragments isolated from adipose tissue to provide an MV/stromal cell suspension; and (b) culturing the MV/stromal cell suspension in an angiogenic medium to provide the functional, vascularized organoid. In embodiments, the stromal cells are mesenchymal stem cells (MSCs) and the suspension is an MV/MSC suspension. In embodiments, said step of culturing comprises incubating the MV/stromal cell suspension or MV/MSC suspension at about 37° C. for at least about 1 day, at least about 2 days, at least about 3 days, at least about 4 days, at least about 5 days, at least about 6 days, at least about 7 days, at least about 8 days, at least about 9 days, at least about 10 days, at least about 15 days, or as needed until a desired level of vascularization is achieved. In a specific embodiment, the culturing comprises incubating the MV/stromal cell suspension or MV/MSC suspension at about 37° C. for at least about 7 days.

In embodiments, the ratio of MVs:stromal cells or MVs:MSCs in the MV/stromal cell suspension or MV/MSC suspension may be adjusted to achieve the desired level of vascularization in the organoid. In embodiment, the ratio of MVs:stromal cells or MVs:MSCs may be about 1:1000, about 1:900, about 1:800, about 1:700, about 1:600, about 1:500, about 1:400, about 1:300, about 1:200, about 1:100, about 1:90, about 1:80, about 1:70, about 1:60, about 1:50, about 1:40, about 1:30, about 1:20, about 1:10, about 1:9, about 1:8, about 1:7, about 1:6, about 1:5, about 1:4, about 1:3, about 1:2, or about 1:1. In embodiments, the ratio of MVs:stromal cells or MVs:MSCs ranges from about 1:1000 to about 1:1, from about 1:100 to about 1:10, or from about 1:100 to about 1:50. In a specific embodiment, the ratio of MVs to stromal cells or MVs to MSCs is about 1:50.

In embodiments, the angiogenic medium is employed to produce organoids comprising undifferentiated stromal cells or MSCs. In embodiments, the angiogenic medium is formulated to comprise Roswell Park Memorial Institute (RPMI) medium, B-27 supplement, fetal bovine serum (FBS), and vascular endothelial growth factor (VEGF). In embodiments, the concentration of FBS ranges from about 0.1% to about 5%, from about 0.1% to about 3%, from about 0.1% to about 1%, from about 0.1% to about 0.9%, from about 0.1% to about 0.8%, from about 0.1% to about 0.7%, from about 0.1% to about 0.6%, from about 0.1% to about 0.5%, from about 0.1% to about 0.4%, from about 0.1% to about 0.3%, or about 0.5%.

In embodiments, the concentration of VEGF ranges from about 1 ng/ml to about 100 ng/ml, from about 1 ng/ml to about 90 ng/ml, from about 1 ng/ml to about 80 ng/ml, from about 1 ng/ml to about 70 ng/ml, from about 1 ng/ml to about 60 ng/ml, from about 1 ng/ml to about 50 ng/ml, from about 1 ng/ml to about 40 ng/ml, from about 1 ng/ml to about 30 ng/ml, or about 50 ng/ml VEGF.

B-27 supplement (Gibco) is a proprietary neuronal cell culture supplement comprising biotin, DL alpha tocopherol acetate, DL alpha tocopherol, vitamin A, bovine serum albumin, catalase, human recombinant insulin, human transferrin, superoxide dismutase, corticosterone, D-galactose, ethanolamine HCl, glutathione, L-carnitine HCl, linoleic acid, linolenic acid, progesterone, putrescine 2HCl, sodium selenite, and trido-l-thryonine (T3). B-27 is available in a 50× concentrated solution, which is diluted per manufacturer's recommendations in the angiogenic medium.

Microvessels are intact microvessel fragments or segments isolated from living tissue. In embodiments, microvessels are harvested and isolated from adipose tissue, particularly human adipose tissue. MVs are native and provide a complete source of microvascular cells, which recapitulate the native vascularization. MVs display phenotypic plasticity and dynamic adaptation under angiogenic conditions, via angiogenesis. In embodiments, MVs for use in the instant organoids and methods may be allogeneic MVs or may be autologous MVs derived from patient tissue, such as adipose tissue. In a specific embodiment, the MVs are Angiomics™ MVs (Advanced Solutions, Louisville, Ky.).

Optionally, the organoids and methods disclosed herein comprise collagen. In embodiments, the MV/stromal cell suspension or the MV/MSC suspension further comprises collagen. In embodiments, the concentration of collagen in the MV/stromal cell suspension or MV/MSC suspension may be about 50%, about 40%, about 30%, about 25%, about 20%, about 15%, about 10%, about 5%, about 4%, about 3%, about 2%, or about 1%. In a specific embodiment, the concentration of collagen in the MV/stromal cell suspension or MV/MSC suspension is about 30%.

In another embodiment, a method for producing a functional, vascularized differentiated adipocyte organoid is provided, the method comprising: (a) culturing mesenchymal stem cells (MSCs) in an adipocyte differentiation medium (ADM) to provide committed pre-adipocyte cells; (b) mixing a suspension of committed pre-adipocyte cells with microvessel (MV) fragments isolated from adipose tissue to provide an MV/pre-adipocyte suspension; and (c) culturing the MV/pre-adipocyte suspension in an adipocyte maintenance medium (AMM) to provide the functional, vascularized adipocyte organoid. Culturing the organoid in stages, using distinct media, permits differentiation of the MSCs as well as the promotion of angiogenesis in the cultured organoid.

In embodiments, the culturing of MSCs in ADM of step (a) is carried out at about 37° C. for a differentiation duration of at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 25 days, or as needed until a desired level of MSC differentiation is achieved. In embodiments, the differentiation duration is selected to permit differentiation of MSCs to committed pre-adipocyte cells. In a very specific embodiment, the differentiation duration is at least about 17 days.

The adipocyte differentiation medium (ADM) promotes the differentiation of MCSs to committed pre-adipocyte cells. In embodiments, the ADM is formulated to comprise Dulbecco's Modified Eagle Medium (DMEM) supplemented with dexamethasone, 3-isobutyl-1-methylxanthine (IBMX), indomethacin, insulin, and fetal bovine serum (FBS).

In embodiments, the concentration of dexamethasone ranges from about 10 μM to about 500 μM from about 10 μM to about 400 μM from about 10 μM to about 300 μM from about 10 μM to about 200 μM from about 10 μM to about 100 μM from about 10 μM to about 90 μM from about 10 μM to about 80 μM from about 10 μM to about 50 μM or about 100 μM.

In embodiments, the concentration of IBMX ranges from about 0.01 mM to about 1 mM, from about 0.1 mM to about 0.9 mM, from about 0.1 mM to about 0.8 mM, from about 0.1 mM to about 0.4 mM, from about 0.1 mM to about 0.3 mM, or about 0.5 mM.

In embodiments, the concentration of indomethacin ranges from about 10 μM to about 500 μM from about 10 μM to about 400 μM from about 10 μM to about 300 μM from about 10 μM to about 200 μM from about 10 μM to about 100 μM from about 10 μM to about from about 10 μM to about 80 μM from about 10 μM to about 50, or about 100 μM.

In embodiments, the concentration of insulin ranges from about 0.5 μg/ml to about 10 μg/ml, from about 1 μg/ml to about 10 μg/ml, from about 1 μg/ml to about 9 μg/ml, from about 1 μg/ml to about 8 μg/ml, from about 1 μg/ml to about 7 μg/ml, from about 1 μg/ml to about 6 μg/ml, from about 1 μg/ml to about 5 μg/ml, from about 2 μg/ml to about 6 μg/ml, or about 5 μg/ml.

In embodiments, the concentration of FBS ranges from about 0.1% to about 5%, from about 0.1% to about 3%, from about 0.1% to about 1%, from about 0.1% to about 0.9%, from about 0.1% to about 0.8%, from about 0.1% to about 0.7%, from about 0.1% to about 0.6%, from about 0.1% to about 0.5%, from about 0.1% to about 0.4%, from about 0.1% to about 0.3%, or about 0.5%.

In embodiments, the ratio of MVs:pre-adipocytes in the MV/pre-adipocyte suspension may be adjusted to achieve the desired level of vascularization in the organoid. In embodiment, the ratio of MVs:MSCs may be about 1:1000, about 1:900, about 1:800, about 1:700, about 1:600, about 1:500, about 1:400, about 1:300, about 1:200, about 1:100, about 1:90, about 1:80, about 1:70, about 1:60, about 1:50, about 1:40, about 1:30, about 1:20, about 1:10, about 1:9, about 1:8, about 1:7, about 1:6, about 1:5, about 1:4, about 1:3, about 1:2, or about 1:1. In embodiments, the ratio of MVs:pre-adipocytes ranges from about 1:1000 to about 1:1, from about 1:100 to about 1:10, or from about 1:100 to about 1:50. In a specific embodiment, the ratio of MVs to pre-adipocytes is about 1:50.

In embodiments, the culturing of the MV/pre-adipocyte suspension in AMM of step (c) is carried out at about 37° C. for at least about 1 day, at least about 2 days, at least about 3 days, at least about 4 days, at least about 5 days, at least about 6 days, at least about 7 days, at least about 8 days, at least about 9 days, at least about 10 days, at least about 15 days, or as needed until a desired level of vascularization is achieved. In a specific embodiment, the culturing comprises incubating the MV/pre-adipocyte suspension at about 37° C. for at least about 7 days.

The adipocyte maintenance medium (AMM) promotes angiogenesis of the organoid comprising committed pre-adipocyte cells. In embodiments, the AMM is formulated to comprise RPMI, DMEM, B-27 supplement, insulin, indomethacin, and fetal bovine serum (FBS).

In embodiments, the concentration of RPMI ranges from about 30% to about 70%, optionally about 50%. In embodiments, the concentration of DMEM ranges from about 30% to about 70%, optionally about 50%. In a specific embodiment, the ratio of RPMI:DMEM is about 50:50.

In embodiments, the concentration of insulin ranges from about 0.5 μg/ml to about 10 μg/ml, from about 1 μg/ml to about 10 μg/ml, from about 1 μg/ml to about 9 μg/ml, from about 1 μg/ml to about 8 μg/ml, from about 1 μg/ml to about 7 μg/ml, from about 1 μg/ml to about 6 μg/ml, from about 1 μg/ml to about 5 μg/ml, from about 2 μg/ml to about 6 μg/ml, or about 5 μg/ml.

In embodiments, the concentration of indomethacin ranges from about 10 μM to about 500 μM from about 10 μM to about 400 μM from about 10 μM to about 300 μM, from about 10 μM to about 200 μM from about 10 μM to about 100 μM from about 10 μM to about 90 μM, from about 10 μM to about 80 μM from about 10 μM to about 50 μM, or about 100 μM.

In embodiments, the concentration of FBS ranges from about 0.1% to about 5%, from about 0.1% to about 3%, from about 0.1% to about 1%, from about 0.1% to about 0.9%, from about 0.1% to about 0.8%, from about 0.1% to about 0.7%, from about 0.1% to about 0.6%, from about 0.1% to about 0.5%, from about 0.1% to about 0.4%, from about 0.1% to about 0.3%, or about 0.5%.

B-27 supplement (Gibco) is a proprietary neuronal cell culture supplement comprising biotin, DL alpha tocopherol acetate, DL alpha tocopherol, vitamin A, bovine serum albumin, catalase, human recombinant insulin, human transferrin, superoxide dismutase, corticosterone, D-galactose, ethanolamine HCl, glutathione, L-carnitine HCl, linoleic acid, linolenic acid, progesterone, putrescine 2HCl, sodium selenite, and trido-l-thryonine (T3). B-27 is available in a 50× concentrated solution, which is diluted per manufacturer's recommendations in the AMM.

In embodiments, microvessels are harvested and isolated from adipose tissue, particularly human adipose tissue. In a specific embodiment, the MVs are Angiomics™ MVs (Advanced Solutions, Louisville, Ky.).

Optionally, the organoids and methods disclosed herein comprise collagen. In embodiments, the pre-adipocyte suspension further comprises collagen. The concentration of collagen in the pre-adipocyte suspension may be about 50%, about 40%, about 30%, about 25%, about 20%, about 15%, about 10%, about 5%, about 4%, about 3%, about 2%, or about 1%. In a specific embodiment, the concentration of collagen in the pre-adipocyte suspension is about 30%.

### Methods of Screening

In another embodiment, a method of screening a compound for pharmacological or toxicological activity is provided, the method comprising: (a) providing a vascularized organoid or spheroid comprising stromal cells and isolated microvessel (MV) fragments; (b) administering a test compound to the organoid or spheroid; and (c) detecting a pharmacological or toxicological response of the organoid or spheroid. In a specific embodiment, the organoid is an adipocyte organoid according to the present disclosure.

In embodiments, the response that is detected comprises one or more of cell death; cell growth; cell differentiation; change in inosculation of microvessels; change in organoid or spheroid diameter; change in organoid or spheroid size; upregulation or downregulation of production of a biomarker; and change of performance in a functional assay.

In embodiments, the biomarker is a biomarker of healthy adipose tissue selected from the group consisting of adiponectin, peroxisome proliferator-activated receptor gamma (PPAR-γ), and glucose transporter (GLUT4). In embodiments, the biomarker is a biomarker of inflammation selected from the group consisting of interleukin 6 (IL-6), interleukin 1 (IL-1), and tumor necrosis factor alpha (TNF-α). In embodiments, the functional assay is selected from the group consisting of a glucose uptake assay, an insulin signaling assay, and a lipolysis assay.

### EXAMPLES

The following examples are given by way of illustration are not intended to limit the scope of the disclosure.

### Example. 1 Materials and Methods

### Microvessel Isolation

Microvessels were isolated from discarded human lipoaspirates, similarly to previously reported protocols (Shepherd, B. R., et al., Rapid perfusion and network remodeling in a microvascular construct after implantation. Arterioscler Thromb Vasc Biol, 2004. 24(5): 898-904; Hoying, J. B., C. A. Boswell, and S. K. Williams, Angiogenic Potential of Microvessel Fragments Established in Three-Dimensional Collagen Gels. In Vitro Cell. Dev. Biol.-Animal, 1996. 32: 409-19). Briefly, adipose tissue is subjected to a limited collagenase digestion, followed by selective screening to remove remaining pieces of tissue and single cells. Each isolation of microvessels is subject to quality control testing, where angiogenic potential is assessed based on neovessel growth in a given amount of time. To reduce the effects of donor-to-donor variation, only lots with similar angiogenic potentials are used.

### Cell Culture

Bone marrow-derived human mesenchymal stem cells (MSCs) were purchased from Rooster Bio. Cells were expanded in DMEM/F12 (Gibco) with 10% fetal bovine serum (FBS; Gibco) in T75 flasks coated with 0.1% gelatin. After 2 passages, cells are used for MSC organoid formation or differentiated into pre-adipocytes following previously described protocols, with slight modifications. Adipocyte differentiation medium (ADM) contained DMEM supplemented with 100 μM dexamethasone (Sigma), 0.5 mM IBMX (Sigma), 100 μM indomethacin (Sigma), 5 μg/ml insulin (Sigma), and 0.5% FBS. Cells were cultured at 37° C. for 17 days, with medium changed every 2-3 days. On day 17, cells were either used for adipose-like organoid formation or continued in 2D culture. At this point, organoids or cells were switched to adipocyte maintenance medium (AMM). AMM contained 50:50 RPMI:DMEM supplemented with B-27, 5 μg/ml insulin, 100 μM indomethacin, and 0.5% FBS. After 7 days of culturing at 37° C. in AMM, cultures were either fixed, flash frozen, or used for functional assays.

### Transwell Assay

Microvessels were plated in wells of a 24 well plate at a density of 100 k/ml in 3 mg/ml collagen (Corning). MSCs were then trypsinized and resuspended in 3 mg/ml collagen at 900 k cells per ml and pipetted into transwell inserts (270 k cells per well). After the collagen gelled, transwell inserts with MSCs were moved into half of the microvessel-containing wells of the 24 well plate, so microvessel growth with and without microvessels could be compared. Groups were tested in serum free microvessel medium containing DMEM/F12, 10 μg/ml insulin (Sigma), 100 μg/ml transferrin (Sigma), 30 nM sodium selenite (Sigma), 100 μM putrescine (Sigma), 20 nM progesterone (Sigma), and with or without the addition of 50 ng/ml vascular endothelial growth factor (VEGF; Peprotech). Microvessels were cultured at 37° C. for 6 days prior to fixing overnight in 10% neutral buffered formalin (NBF; Fisher Scientific), with a medium change on day 4.

### Organoid Formation

MSCs or pre-adipocytes were trypsinized and resuspended at 2 million cells per ml (50 k cells/organoid in all cases). In experiments containing microvessels, microvessels were mixed with the cell suspension prior to seeding. 25 μl of the combined suspension was seeded per organoid in a non-adherent V-bottom 96 well plate. In experiments evaluating the inclusion of collagen, a 3 mg/ml solution of collagen was prepared, and a volume was added to the cell suspension such that 30% of the total cell suspension volume was collagen. Microvessel incorporation was evaluated at MV:MSC ratios of 1:100, 1:50, 1:25, or 1:12.5. Subsequent experiments were all seeded using the 1:50 ratio (1 k microvessels and 50 k cells per organoid). All undifferentiated MSC organoids were cultured in standard microvessel angiogenic medium, which contains RPMI supplemented with B-27, 0.5% FBS, and 50 ng/ml VEGF. Adipose-like organoids were instead cultured in AMM. Culture medium was changed every 2 days.

### Measuring Angiogenic Potential

Organoids were embedded in 3 mg/ml collagen after 2 or 5 days of culture. During embedded culture, angiogenic microvessels invade the collagen surrounding the organoid. After 2 days, the embedded organoids were fixed overnight in 10% neutral-buffered formalin (NBF). After embedded organoids are cultured and fixed, they are stained with lectin as described below. The number of microvessels growing out of each organoid was counted and normalized to organoid circumference, measured in ImageJ. Organoid diameter was calculated from circumference.

### Lipolysis Assay

A lipolysis assay was performed on 2D (after 24 days of 2D culture) and 3D cultures (after 17 days of 2D and 7 days of 3D culture), using a commercially available kit (Sigma). Samples were treated with 1 μM isoproterenol for 4 hours. For 2D cultures, supernatants were then mixed with the reaction solution and incubated according to manufacturer's instructions. For 3D cultures, organoids were homogenized in the supernatant after isoproterenol incubation using a plastic pestle, to release glycerol trapped inside the organoid. The homogenized sample was briefly centrifuged, and supernatants were used for the remainder of the manufacturer's protocol. Samples were read at 570 nm using a plate reader (BioTek Instruments). For 2D cultures, samples cultured in FBS were used as control samples that will not undergo lipolysis. For 3D samples, negative controls were not treated with isoproterenol.

### Tumor Necrosis Factor Alpha (TNF-α) Challenge

Adipose organoids were seeded and cultured as described above. After 7 days of culture, medium was changed from AMM to AMM supplemented with or without 50 ng/ml TNF-α (Peprotech). The following day, organoids were either flash frozen for PCR analysis, fixed for histology, or used for an IL-6 enzyme-linked immunosorbent assay (ELISA).

### IL-6 ELISA

Organoids with and without microvessels and with or without TNF-α treatment were transferred with their supernatants to micro-centrifuge tubes and briefly homogenized with a plastic pestle. Samples were diluted and centrifuged for 2 minutes at 12,000 rcf to pellet remaining matrix and cell debris. An ELISA was performed with the supernatant following manufacturer instructions (R&D systems) to measure secreted interleukin-6 (IL-6) as a marker of adipocyte inflammation and dysfunction.

### Histology and Staining

3D constructs were fixed overnight in 10% NBF at 4° C., then permeabilized for 30 minutes with 0.25% Triton X-100, blocked overnight at 4° C. in 5% bovine serum albumin (BSA; Fisher Scientific), and stained overnight at 4° C. in a 1:50 dilution of rhodamine labelled Ulex Europaeus Agglutinin I (UEA I) lectin (Vector Laboratories) in 5% BSA. After 30 minutes in Hoechst dye (1:3000 in DI water) at room temperature, constructs were washed a minimum of 4 times, with at least one overnight wash. Imaging was performed using a confocal Olympus FV3000 (organoid experiments) or an IN Cell 6500 high content analysis scanner (Cytiva; transwell experiment). Additional phase contrast images were taken of transwell constructs with an Olympus CKX53 inverted microscope.

Additional fixed organoids were stained with a BODIPY™ 493/503 (4,4-Difluoro-1,3,5,7,8-Pentamethyl-4-Bora-3a,4a-Diaza-s-Indacene) dye (Invitrogen). After permeabilization and blocking as described above, dye was added to organoids at a 1:100 dilution in 5% BSA. After 30 minutes, organoids were rinsed multiple times with PBS and imaged on an IN Cell analyzer 6500 HS (Cytiva).

Fixed, non-collagen embedded organoids were processed, paraffin embedded, and sectioned (Saffron Scientific). Slides were deparaffinized and rehydrated to water prior to staining with Harris Hematoxylin (Sigma) and Eosin (Sigma) to visualize morphology or picrosirius red (Sigma) to visualize collagen with fast-green counterstain (Sigma).

2D cell cultures were fixed for 30 minutes at room temperature in 10% NFB, incubated with 60% isopropyl alcohol for 5 minutes, and then Oil Red 0 (Sigma) for 15 minutes. Samples were rinsed thoroughly in 60% isopropyl alcohol and then water. A Meyer's Hematoxylin stain (Sigma) was used for 15 minutes followed by additional PBS washes and imaging on an Olympus CKX53 microscope.

### Image Analysis

Phase contrast images of microvessels used for the transwell assay were analyzed using the BioSegment™ Software Application (Advanced Solutions). The software uses machine learning to identify and quantify microvessel length in an image. Briefly, a fraction of images was annotated manually, wherein the user identified and marked individual vessels. From this information, the program “learned” the identifying features of a microvessel and accurately identified vessels from the remainder of the images and provided microvessel length for each image. Lengths calculated from each image (minimum of 6 per well) within a single well were added together, to yield a length value for each well. Three wells were averaged for each group, to provide average microvessel length under each treatment condition.

### Polymerase Chain Reaction (PCR)

RNA was extracted from either 2D cultures or 3D organoids using a Qiagen RNeasy Plus Micro kit following manufacturer's protocols. RNA was converted to cDNA with SuperScript™ IV VILO™ Master Mix (Fisher Scientific). cDNA was amplified with human primers (IDT Technologies) listed in Table 1. Primers for insulin receptor were designed using Primer3 software (NIH) and verified using BLAST database (NIH), with gene coding obtained from GenBank (NIH).

TABLE 1
Primer sets used for PCR

| Gene | Primer Sequence | |
|---|---|---|
| Adiponectin | 5′-AAGGAGATCCAGGTC TTATTGG-3′ | SEQ ID NO: 1 |
| Forward | | |
| Adiponectin | 5′-ACCTTCAGCCCCGGGTAC-3′ | SEQ ID NO: 2 |
| Reverse | | |
| PPAR-γ Forward | 5′-CATAAAGTCCTTCCCGCTGA-3′ | SEQ ID NO: 3 |
| PPAR-γ Reverse | 5′-GGGGGTGATGTGTTTGAACT-3′ | SEQ ID NO: 4 |
| IL-6 Forward | 5′-GGTACATCCTCGACGGCATCT-3′ | SEQ ID NO: 5 |
| IL-6 Reverse | 5′-GTGCCTCTTTGCTGCTTTCAC-3′ | SEQ ID NO: 6 |
| Insulin Receptor | TGCAAACCCAAGAACGTCAG | SEQ ID NO: 7 |
| Forward | | |
| Insulin Receptor | AGCTTCCGGGAGTTCAGTAC | SEQ ID NO: 8 |
| Reverse | | |

Real-time PCR was performed using a SYBER master mix (Fisher Scientific) and CFX96 Real Time System (Bio Rad). Adiponectin and PPAR-γ expression were normalized to GAPDH (IDT Technologies) and compared to either FBS treated controls (2D) or organoids without microvessels (3D). After RT-PCR, amplified cDNA was loaded into a 2% agarose gel and run at 90V. Gels were imaged for SYBER green on a gel scanner (Azure Biosystems).

### Statistical Analysis

Statistics were performed using SigmaPlot 11.0 (Systat). Where applicable, one-way ANOVA tests were performed with a Newman-Keuls post hoc analysis or a student's t test. For all comparisons, the significance level α=0.05.

### Example 2. Mesenchymal Stem Cells Promote Angiogenesis

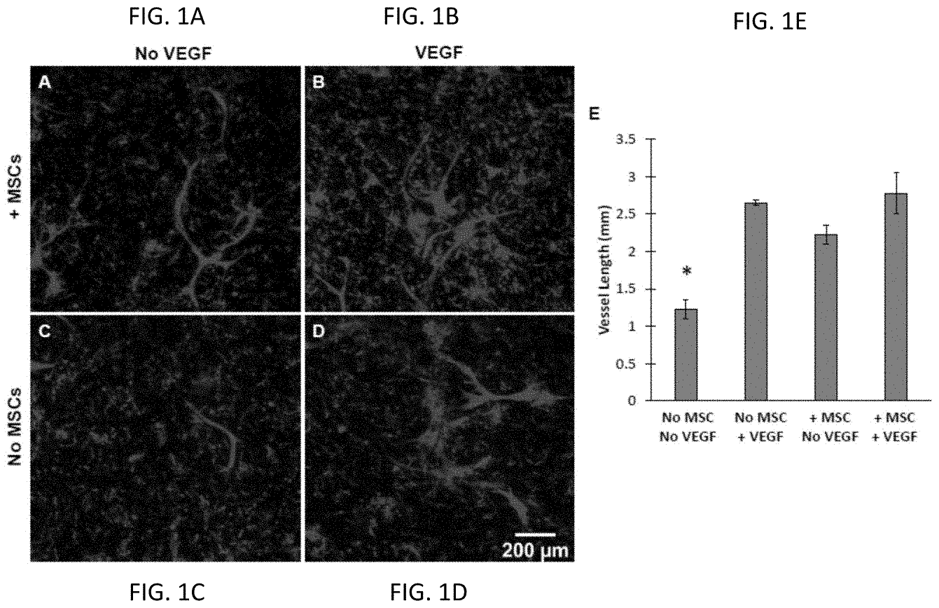

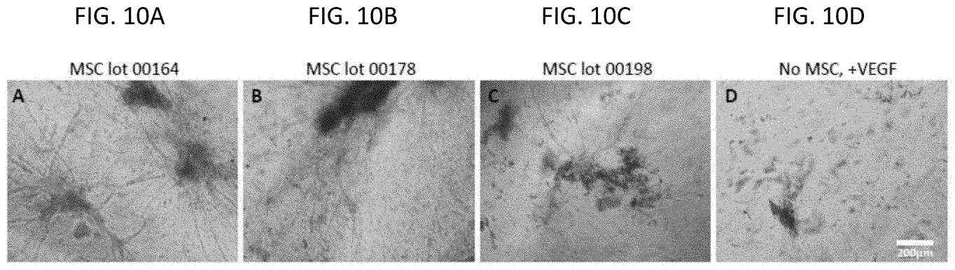

To evaluate the effect of MSCs on angiogenesis from the isolated microvessels, MSCs were embedded in collagen and placed on top of a transwell insert, while microvessels were cultured in collagen on the well plate surface, below the insert. Groups were tested in a standard serum-free medium used to culture human microvessels, both with and without exogenous VEGF. Microvessels cultured with either MSCs or VEGF had significantly increased vascular growth (FIGS. 1A-1D). The inclusion of MSCs increased vessel density comparably to exogenous VEGF alone (FIG. 1E). Additional lots of MSCs from different donors subjectively yielded similar increases in microvessel growth when compared to controls without MSCs (FIG. 10).

Isolated microvessels and MSCs were cultured in the same well using a transwell insert, which physically separates the MSCs and microvessels, but allows molecule exchange between the respective media compartments. MSCs secrete a host of angiogenic growth factors, including but not limited to VEGF, FGF, IGF, and HGF. Thus, groups with MSCs produced a higher vessel density than groups without MSCs. This effect was comparable to exogenous high concentrations of VEGF (50 ng/ml), which is typically used to stimulate angiogenesis (FIGS. 1A-1E). This lot of MSCs produced 467 pg/10⁵ cells per day of VEGF (as determined by the manufacturer). After 4 days of culture, assuming a 24-hour MSC doubling time, 3.72 ng of VEGF is produced in 1.8 ml of medium, which is substantially less than in groups treated with exogenous VEGF. This indicates that the combination of multiple growth factors released into the medium by the MSCs is more effective than high doses of VEGF alone. This may have larger implications for development of future microvessel-containing tissues, as many tissue systems may not respond well to high doses of growth factors. For example, VEGF is inhibitory of adipocyte differentiation. If MSCs can be added to a tissue construct and produce comparable or improved angiogenesis over VEGF, it widens the number of potential applications for the microvessel vascularization system.

### Example 3. Microvessels Form Vascular Networks within MSC Organoids

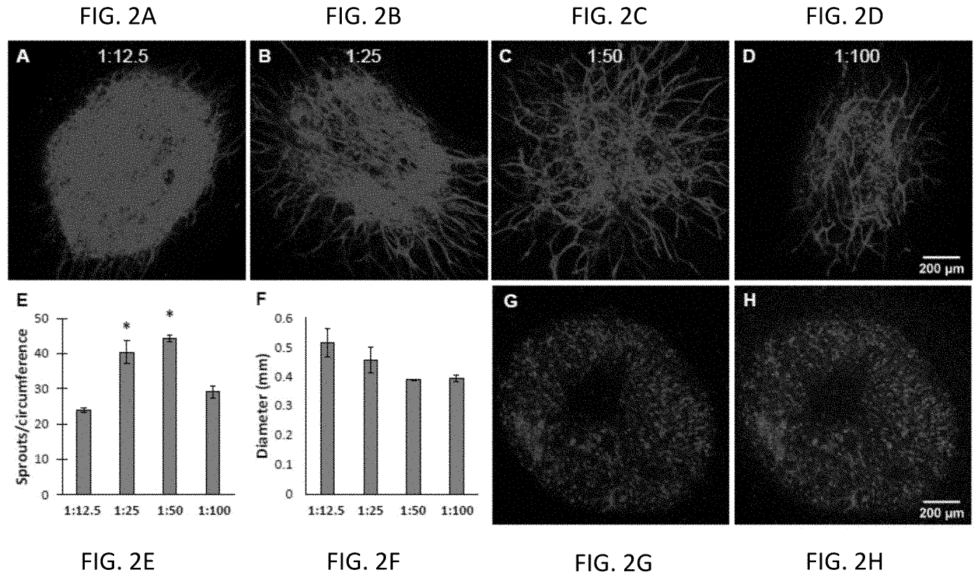

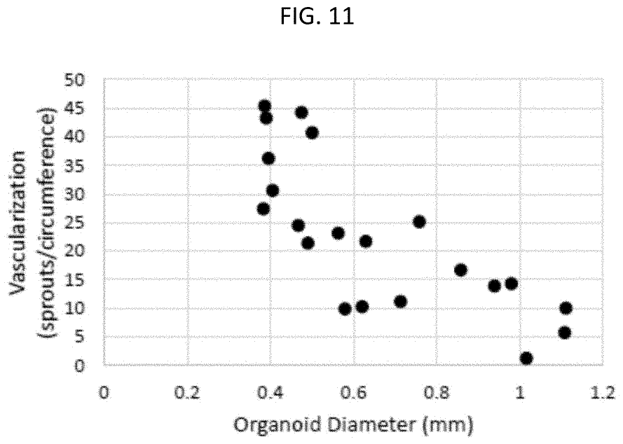

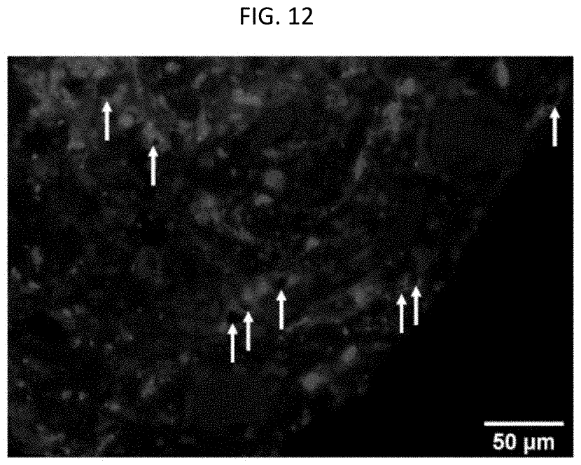

Microvessels were mixed with MSCs to create organoids at ratios of either 1:100, 1:50, 1:25, or 1:12.5 MVs:MSCs. Organoids embedded in collagen at day 5 and fixed on day 7 showed robust angiogenic outgrowth and network formation (FIGS. 2A-2D). The number of neovessel sprouts growing out of each organoid into the surrounding matrix was quantified and normalized to organoid circumference (FIGS. 2E-2F). Organoids with the highest concentration of microvessels (1:12.5) were challenging to image, as most of the organoid appeared to consist of lectin positive cells, making it impossible to distinguish vessels within the organoid. Outgrowths in the 1:12.5 group were subjectively shorter than those in the other groups, and these organoids had the largest diameter (FIG. 2F). Both the 1:100 and 1:12.5 had significantly lower numbers of angiogenic outgrowths than 1:50 or 1:25. Between the 1:50 and 1:25 groups, the vascular network in the 1:50 ratio appeared more distinct throughout, with longer outgrowths. Organoids embedded at an earlier time point, day 2, showed limited angiogenic growth when analyzed at day 4, although incorporated fragments are still visible (FIGS. 2G, 2H; 1:50 group shown). This was not surprising, as microvessels embedded in collagen typically do not show robust growth until around days 5-7. Interestingly, some correlation was observed between organoid size and vascular potential, suggesting that the lower angiogenic potential in organoids with more microvessels may be due, in part, to their larger size (squared correlation coefficient=0.597, FIG. 11). A cross section of the organoid is shown in FIG. 12, where vessel lumens can be seen throughout the construct.

Visualizing angiogenesis within the organoids was challenging due to difficulties in distinguishing neovessels within the organoid. A functional assay was developed to assess angiogenic potential of the microvessels, as an indicator of microvessel presence and function, within the organoids. The organoids were embedded in a collagen matrix and angiogenic sprouts growing out of the organoids into the matrix were quantified. Within 48 hours, rapid vessel outgrowth was observed from the vessels into the surrounding collagen (FIGS. 2A-2H). In addition to demonstrating functional integrity of the neovasculatures in the organoids, the robust outgrowth of neovessels into the surrounding matrix suggests that, if implanted, these organoids will rapidly inosculate with the surrounding host circulation.

Angiogenic growth is relative to the ratio of MVs:MSCs within the organoid. When microvessels were seeded at higher numbers, fewer angiogenic sprouts were observed crossing into the collagen matrix (FIGS. 2A-2H). Comparing organoid vascularity and diameter suggests an optimal size threshold of 400 μm: increasingly larger organoids above this threshold exhibit limited vascularization while organoids below this threshold, regardless of size, supported vascularization.

While not desiring to be bound by theory, it is believed that larger organoids experience necrosis due to limitations in oxygen and nutrient diffusion. Given that the average diameter of the 1:12.5 group was approximately 500 μm, some functionality may have been lost due to necrosis. The 1:50 and 1:100 groups measured under 400 μm, while the 1:25 group measured above this threshold. Both the 1:50 and 1:100 groups had comparable numbers of sprouts growing out the organoid, although 1:50 appeared to have a more robust vascular network throughout. The 1:100 had fewer sprouts, likely due to the overall lower number of microvessels. Vascularity and diameter of organoids were plotted from all MSC experiments on a single graph (FIG. 11). Results indicated a correlation, particularly above 500 μm, although an R² value of 0.597 suggests it is likely not the sole contributing factor.

### Example 4. Collagen is not Necessary for Vascular Network Formation in Organoids

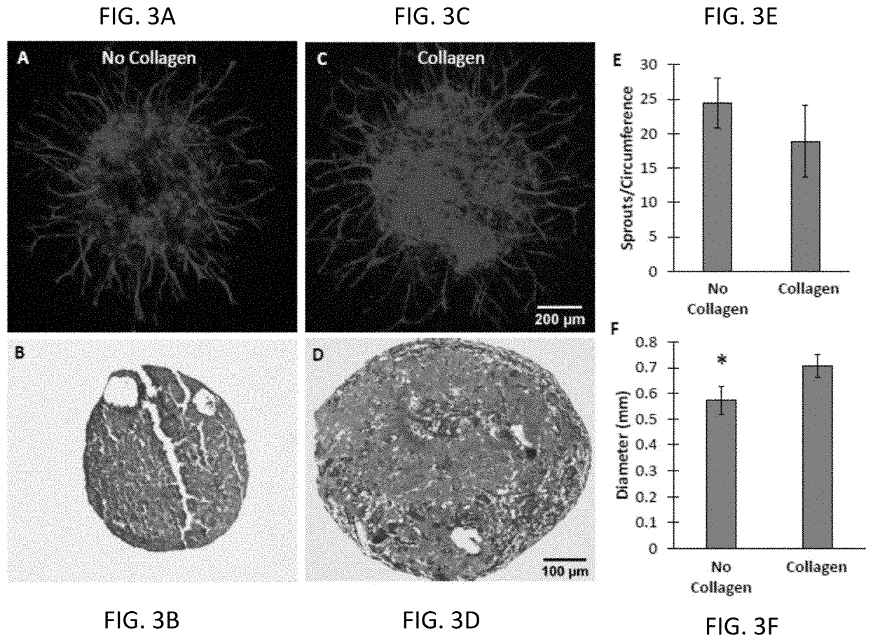

Initially, collagen was included in organoid fabrication protocols, as previously observed that microvessels need a fibrillar matrix to grow and survive. However, organoid fabrication without collagen was explored in efforts to increase cell densities. Compared to organoids made without collagen (FIGS. 3A, 3B), the presence of collagen (FIGS. 3C, 3D) did not have a significant effect on angiogenic growth (FIG. 3E). Picrosirius red/fast green staining of organoid sections showed that collagen remained in collagen-formed organoids after 7 days of culture, although its distribution was uneven throughout the organoid (FIGS. 3B, 3D). This likely contributed to overall larger diameters observed in organoids with collagen (FIG. 3F). In this group, there is a small amount of collagen around the organoid edges that is much less dense and likely secreted by cells. Interestingly, however, less of this cell-secreted collagen was visible in organoids where collagen was not included in initial fabrication.

While not desiring to be bound by theory, it was hypothesized that collagen incorporation would help maintain microvessels until MSCs secreted their own collagen and remodeled the organoid microenvironment. In all experiments, seeded cell/collagen suspensions rapidly contracted into a tight organoid within 24 hours. However, the addition of collagen to an organoid may not be feasible in all cell systems, particularly with cells that do not rapidly remodel collagen. A formal comparison of MSC organoids with and without collagen was thus undertaken. Surprisingly, robust angiogenesis and neovascular network formation occurred whether collagen was included or not (FIGS. 3A-3F). The results indicate that other matrix components provide sufficient structure to support microvessel growth. After 7 days of culture, collagen was still clearly visible in histological sections of the organoids, although it was largely in clumps that were unevenly distributed throughout the organoid. Thus, while some remodeling is evident, MSCs did not completely remodel the initially seeded collagen within the 7-day period. Almost no collagen was observed in organoids that did not contain collagen in the initial seeding suspension, suggesting that limited collagen is being produced by MSCs. Overall, the results indicate that microvessels are capable of robust angiogenesis with or without collagen in organoids.

### Example 5. MSC Derived Pre-Adipocytes can be Maintained in Angiogenic Medium

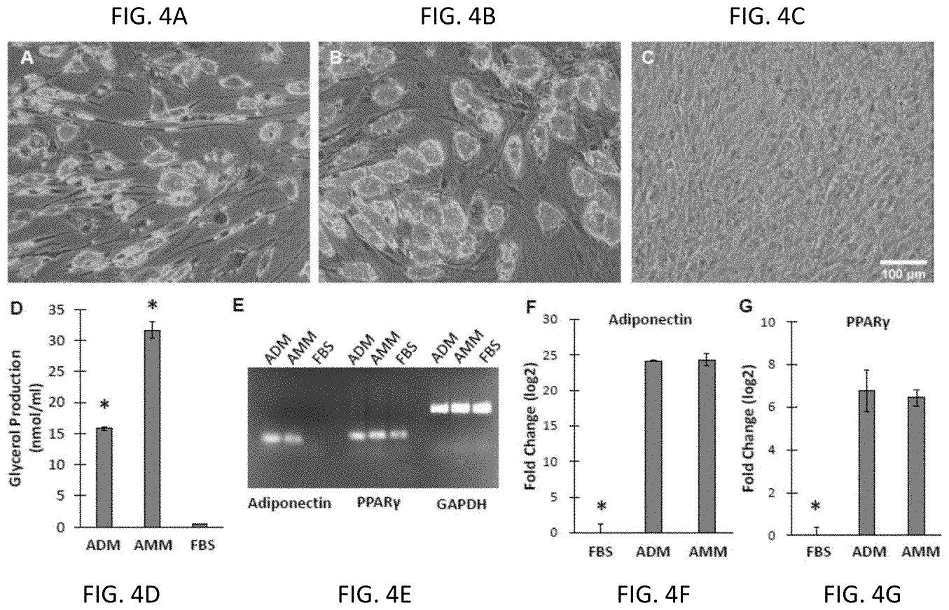

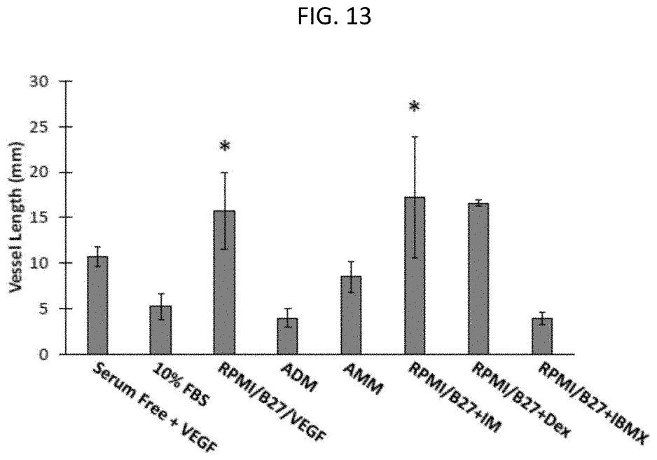

Toward fabricating adipocyte-organoids, a protocol was developed that employs staged culture media to support differentiation of MSCs into pre-adipocytes while promoting microvessel growth. This protocol, and development of AMM, was necessary, as standard ADM did not support microvessel growth (FIG. 13). In this protocol, MSCs are cultured in 2D with ADM to induce differentiation towards adipocytes. Then, these committed MSCs are combined with isolated microvessels to form the organoid. Organoids are then cultured in AMM, which supported both MSC differentiation and microvessel angiogenesis. To verify that AMM maintains an adipogenic phenotype, some wells after 17 days were switched to AMM for an additional 7 days, while others were maintained in ADM. Subjectively, more lipid droplets were visible after culture in AMM, compared to 24 days in ADM (FIGS. 4A, 4B). Control MSCs cultured in FBS expansion medium had no lipid droplets visible (FIG. 4C). A lipolysis assay was performed at day 24 as a measure of adipocyte function. Cells cultured in AMM after ADM commitment had a significantly higher glycerol release than cultures continued on ADM or on FBS-containing medium (FIG. 4D). Real-time PCR was performed to measure relative expression of adiponectin and PPAR-γ, markers typically associated with mature, functional adipocytes. Adiponectin was expressed in cells cultured in ADM and AMM, but not FBS (FIG. 4E). RT-PCR yielded nearly identical delta Cq values for ADM and AMM, suggesting comparable upregulation in both groups compared to FBS (FIG. 4F). PPAR-γ was expressed in all three groups (FIG. 4E), although PPAR-γ expression was substantially upregulated in both ADM and AMM when compared to FBS controls (FIG. 4G).

In preliminary studies, it was observed that IBMX, which is commonly used to induce pre-adipocyte differentiation, permanently impeded microvessel growth (FIG. 13). Thus, we developed a protocol with two different medium types that are used in stages. An induction medium (ADM) was used to stimulate MSC differentiation to pre-adipocytes and a maturation, or angiogenic medium (AMM) was used to maintain and continue their differentiation while supporting microvessel growth. Here, a medium comprising both insulin and a standard B-27 supplement in an RPMI:DMEM 50:50 mix was employed. B-27 and RPMI strongly support microvessel growth (FIG. 13), so these supplements were combined with DMEM and insulin, which support pre-adipocyte differentiation. This new medium, AMM, resulted in microvessel growth comparable to the serum free medium control (FIG. 13). With the staged treatment protocol, cells had more, and larger lipid droplets visualized with Oil Red 0 staining than ADM treatment alone. Additionally, cells treated with AMM produced higher glycerol amounts when stimulated with isoproterenol (FIGS. 4A-4G). These results suggest that the disclosed staged medium treatments differentiate cells further towards mature adipocytes than ADM alone.

### Example 6. Vascularized Adipose-Like Organoids can be Formed from MSC Derived Pre-Adipocytes

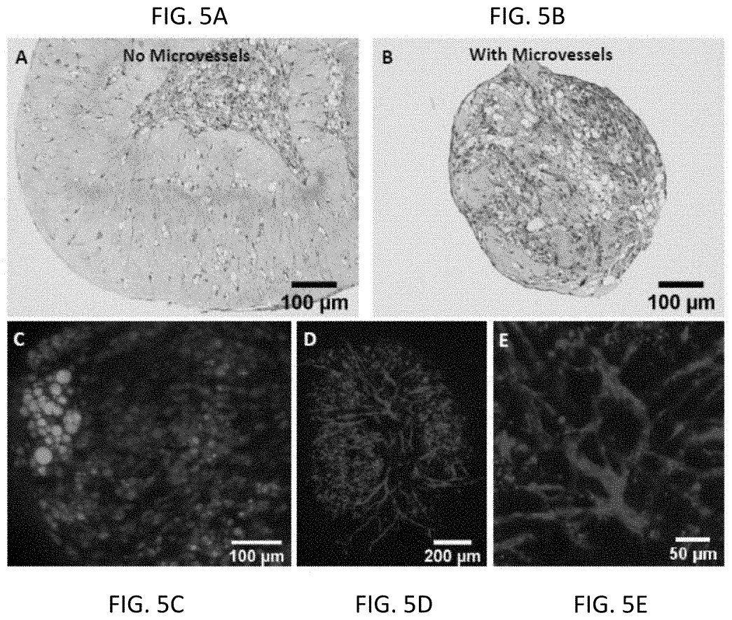

Adipose-like organoids were formed with MSCs that had differentiated for 17 days in ADM with or without the inclusion of microvessels at the time of organoid formation. In initial experiments, collagen was included in organoid fabrication. Organoids were then cultured for 7 days in AMM (with some embedded in collagen on day 5). Paraffin sections stained for hematoxylin and eosin (H&E) suggested that cells had differentiated into mature adipocytes due to the many voids in the tissue where lipid droplets were extracted during processing (FIG. 5A, 5B). Mature adipocytes were also visible in images of whole organoids stained with BODIPY dye (FIG. 5C). Cells in organoids without microvessels did not remodel the collagen, but instead separated from the collagen into cell-dense regions leading to larger organoids (FIG. 5A). Interestingly, in organoids with microvessels, the collagen was remodeled, resulting in a compact, cell dense organoid (FIG. 5B). Still, voids from large lipid droplets are visible, primarily in the organoid center but also spread throughout the collagen. Lectin staining of embedded, whole adipocyte organoids showed highly branched vascular networks growing throughout organoids with incorporated microvessels. When the organoids were placed in a collagen bed, vessel growth was largely contained within the adipocyte organoids, with fewer numbers of angiogenic sprouts invading the surrounding collagen matrix than in MSC experiments (FIGS. 5D, 5E).

Results showed that organoids with microvessels remodeled and compacted collagen, but organoids without microvessels did not. Instead, cells seemed to largely separate out from the collagen, giving the appearance that the collagen was encasing the organoid, with only a small number of cells throughout the collagen (FIGS. 5A-5E). This resulted in extremely large organoid diameters (>800 μm), which may limit oxygen and nutrient diffusion to the center of the organoids and impair function. Because of this, the experiment was repeated without collagen, and these replicates were used for all functional testing and PCR. Interestingly, it was observed that when collagen was included in adipose-like organoids, microvessels grew throughout the organoid and adopted a more mature morphology. This can be clearly seen in FIGS. 5A-5E, where vessels are wider, contain more branch points, and are more interconnected than those seen in MSC organoids (FIGS. 2A-2H).

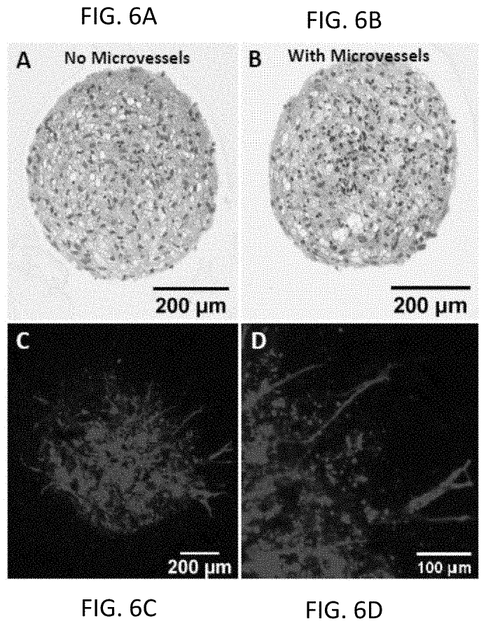

The experiment was repeated without the inclusion of collagen in organoid fabrication as it was unknown whether the large diameter of organoids with collagen and no microvessels would adversely affect adipocyte function, due to diffusion limitations, independently of microvessel inclusion. Morphologically organoids without collagen without and with microvessels looked similar (FIGS. 6A, 6B). Microvessels can be seen growing out of embedded organoids (FIG. 6D, 6E), although they are fewer in number and have a thinner neovessel morphology than with collagen inclusion. Organoids without collagen were used in subsequent PCR and functional tests.

Results showed a limited number of microvessels growing out of the organoid, and those present had a thin, sprout-like morphology that is much less mature (FIGS. 6A-6D). These findings contrasted with earlier experiments in the MSC organoids, where the presence of collagen did not affect microvessel outgrowth (FIGS. 3A-3F). This difference may reflect the pro-angiogenic environment established by the MSCs versus a more stable environment established by more mature adipocytes. It should also be noted that microvessel donor-to-donor variation cannot be completely excluded as different microvessel lots were used in these experiments (despite microvessel qualification tests).

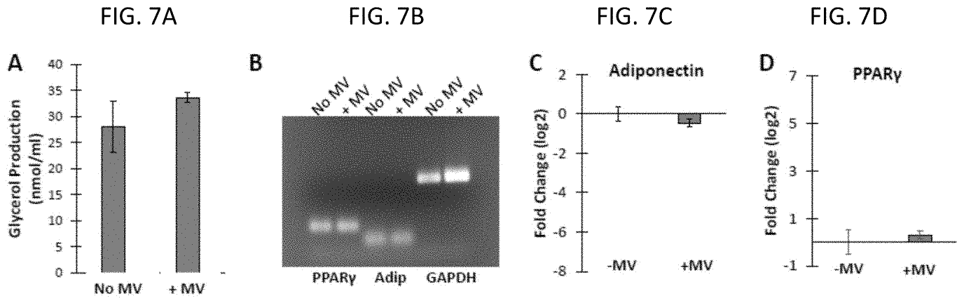

Organoids with and without microvessels produced glycerol in response to isoproterenol, a measure of lipolysis. Those with microvessels produced slightly more glycerol, although this difference was not significant (FIG. 7A). Both groups of organoids expressed the adipocyte markers adiponectin and PPAR-γ (FIG. 7B). RT-PCR showed no meaningful difference in expression of these markers in organoids with microvessels when compared to organoids without microvessels (FIGS. 7C, 7D).

### Example 7. Adipocytes Upregulate Insulin Receptors in the Presence of Microvessels

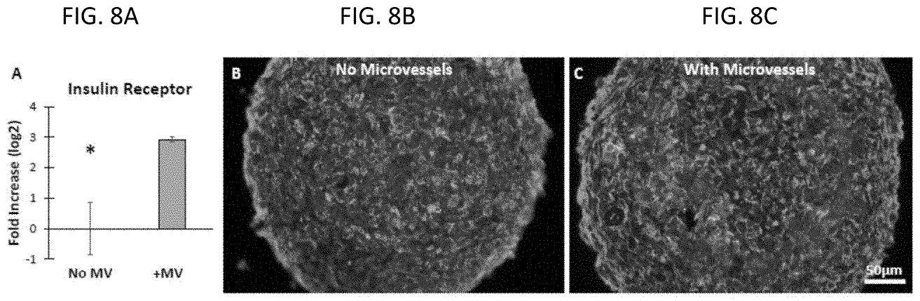

Insulin exerts important functional control of adipocytes, signaling via the insulin receptor. To further evaluate the effect of microvessels on adipocyte function, PCR was performed to examine insulin receptor expression. Organoids with microvessels had a 3-fold (log 2) increase in insulin receptor expression compared to organoids without microvessels (FIG. 8A). Insulin receptor expression occurred primarily on the surface of adipocytes throughout the construct with the included microvessels showing relatively low insulin receptor expression. (FIG. 8B).

### Example 8. Microvessels Modulate Adipose-Like Organoid Secretion of IL-6 in Response to TNF-α

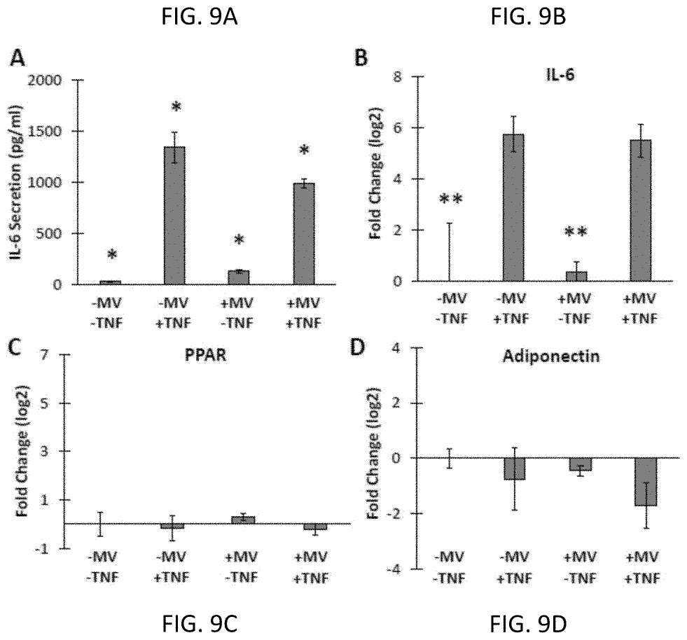

Adipose inflammation, often modeled with exposure to TNF-α, is a hallmark feature of adipose dysfunction. To explore this, adipose-like organoids with and without microvessels were treated with TNF-α for approximately 24 hours followed by measurement of IL-6, an inflammatory cytokine known to increase in response to TNF-α treatment in adipocytes. Without TNF-α, IL-6 was almost nonexistent in samples without microvessels but was present at low levels in organoids with microvessels. Organoids treated with TNF-α had dramatically increased IL-6 secretion, although less was secreted by organoids with microvessels than those without (FIG. 9A). Interestingly, IL-6 expression in both TNF-α treated groups was comparable, suggesting the microvessels caused a difference in IL-6 secretion (FIG. 9B).

In contrast, PPAR-γ expression was not affected by the presence of microvessels when microvessels were present in response to TNF-α (FIG. 9C). Adiponectin expression changed minimally in groups with no microvessels in response to TNF-α and with microvessels and no TNF-α. A small but distinct downregulation was visible in organoids with microvessels following TNF-α treatment (FIG. 9D).

Embodiments can be described with reference to the following numbered clauses, with preferred features laid out in dependent clauses.

1. A method for producing a functional, vascularized organoid, the method comprising: (a) mixing a suspension of stromal cells with microvessel (MV) fragments isolated from adipose tissue to provide an MV/stromal cell suspension; and (b) culturing the MV/stromal cell suspension in an angiogenic medium to provide the functional, vascularized organoid.
2. The method according to clause 1, wherein the stromal cells are mesenchymal stem cells (MSCs) and the MV/stromal cell suspension is an MV/MSC suspension.
3. The method according to clause 2, wherein the ratio of MVs:MSCs in the MV/MSC suspension of step (a) ranges from about 1:100 to about 1:10.
4. The method according to clause 3, wherein the ratio of MVs:MSCs is about 1:50.
5. The method according to any of the preceding clauses, wherein the angiogenic medium comprises Roswell Park Memorial Institute (RPMI) medium, B-27 supplement, fetal bovine serum (FBS), and vascular endothelial growth factor (VEGF).
6. The method according to any of clauses 2-5, wherein the MVs are isolated from human adipose tissue.
7. The method according to any of clauses 2-6, wherein culturing comprises incubating the MV/MSC suspension at about 37° C. for at least about 7 days.
8. The method according to any of clauses 2-7, wherein the suspension of MSCs further comprises collagen.
9. The method according to clause 8, wherein the volume of collagen in the suspension of MSCs is about 30%.
10. A functional, vascularized organoid produced according to the method of any of clauses 1-9.
11. A method for producing a functional, vascularized adipocyte organoid, the method comprising: (a) culturing mesenchymal stem cells (MSCs) in an adipocyte differentiation medium (ADM) to provide committed pre-adipocyte cells; (b) mixing a suspension of committed pre-adipocyte cells with microvessel (MV) fragments isolated from adipose tissue to provide an MV/pre-adipocyte suspension; and (c) culturing the MV/pre-adipocyte suspension in an adipocyte maintenance medium (AMM) to provide the functional, vascularized adipocyte organoid.
12. The method according to clause 11, wherein the ratio of MVs:pre-adipocytes in the MV/pre-adipocyte suspension ranges from about 1:100 to about 1:10.
13. The method according to any of clauses 11-12, wherein the ratio of MVs:pre-adipocytes is about 1:50.
14. The method according to any of clauses 11-13, wherein the adipocyte differentiation medium comprises Dulbecco's Modified Eagle Medium (DMEM), dexamethasone, 3-isobutyl-1-methylxanthine (IBMX), indomethacin, insulin, and fetal bovine serum (FBS).
15. The method according to any of clauses 11-14, wherein the MVs are isolated from human adipose tissue.
16. The method according to any of clauses 11-15, wherein the culturing of step (a) comprises incubating the MSCs in the adipocyte differentiation medium at about 37° C. for at least about 17 days.
17. The method according to any of clauses 11-16, wherein the adipocyte maintenance medium comprises RPMI, DMEM, B-27 supplement, insulin, indomethacin, and FBS.
18. The method according to any of clauses 11-17, wherein the culturing of step (c) comprises incubating the MV/pre-adipocyte suspension in the adipocyte maintenance medium at about 37° C. for at least about 7 days.
19. The method according to any of clauses 11-18, wherein the suspension of committed pre-adipocyte cells further comprises collagen.
20. The method according to clause 19, wherein the volume of collagen in the suspension of committed pre-adipocyte cells is about 30%.
21. A functional, vascularized adipocyte organoid produced according to the method of any of clauses 11-20.
22. A method of screening a compound for pharmacological or toxicological activity, the method comprising: (a) providing a vascularized organoid or spheroid comprising stromal cells and isolated microvessel (MV) fragments; (b) administering a test compound to the organoid or spheroid; and (c) detecting a pharmacological or toxicological response of the organoid or spheroid.
23. The method according to clause 22, wherein the organoid is an adipocyte organoid.
24. The method according to any of clauses 22-23, wherein the response comprises one or more of cell death; cell growth; cell differentiation; change in inosculation of microvessels; change in organoid or spheroid diameter; change in organoid or spheroid size; upregulation or downregulation of production of a biomarker; and change of performance in a functional assay.
25. The method according to any of clauses 22-24, wherein the response comprises upregulation or downregulation of production of a biomarker selected from the group consisting of adiponectin, PPAR-γ, GLUT4, IL-6, IL-1, and TNF-α.
26. The method according to any of clauses 24-25, wherein the functional assay is selected from the group consisting of a glucose uptake assay, an insulin signaling assay, and a lipolysis assay.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

The foregoing description is illustrative of particular embodiments of the invention but is not meant to be a limitation upon the practice thereof. While particular embodiments have been illustrated and described, it would be obvious to one skilled in the art that various other changes and modifications can be made without departing from the spirit and scope of the invention. It is therefore intended to cover in the appended claims all such changes and modifications that are within the scope of this invention.

We claim:

1. A method for producing a functional, vascularized organoid, the method comprising:
(a) mixing a suspension of stromal cells with microvessel (MV) fragments isolated from adipose tissue to provide an MV/stromal cell suspension; and
(b) culturing the MV/stromal cell suspension in an angiogenic medium to provide the functional, vascularized organoid.

2. The method according to claim 1, wherein the stromal cells are mesenchymal stem cells (MSCs) and the MV/stromal cell suspension is an MV/MSC suspension.

3. The method according to claim 2, wherein the ratio of MVs:MSCs in the MV/MSC suspension of step (a) ranges from about 1:100 to about 1:10.

4. The method according to claim 3, wherein the ratio of MVs:MSCs is about 1:50.

5. The method according to claim 1, wherein the angiogenic medium comprises Roswell Park Memorial Institute (RPMI) medium, B-27 supplement, fetal bovine serum (FBS), and vascular endothelial growth factor (VEGF).

6. The method according to claim 1, wherein the MVs are isolated from human adipose tissue.

7. The method according to claim 2, wherein culturing comprises incubating the MV/MSC suspension at about 37° C. for at least about 7 days.

8. The method according to claim 2, wherein the suspension of MSCs further comprises collagen.

9. The method according to claim 8, wherein the volume of collagen in the suspension of MSCs is about 30%.

10. A functional, vascularized organoid produced according to the method of claim 1.

11. A method for producing a functional, vascularized adipocyte organoid, the method comprising:
(a) culturing mesenchymal stem cells (MSCs) in an adipocyte differentiation medium (ADM) to provide committed pre-adipocyte cells;
(b) mixing a suspension of committed pre-adipocyte cells with microvessel (MV) fragments isolated from adipose tissue to provide an MV/pre-adipocyte suspension; and
(c) culturing the MV/pre-adipocyte suspension in an adipocyte maintenance medium (AMM) to provide the functional, vascularized adipocyte organoid.

12. The method according to claim 11, wherein the ratio of MVs:pre-adipocytes in the MV/pre-adipocyte suspension ranges from about 1:100 to about 1:10.

13. The method according to claim 12, wherein the ratio of MVs:pre-adipocytes is about 1:50.

14. The method according to claim 11, wherein the adipocyte differentiation medium comprises Dulbecco's Modified Eagle Medium (DMEM), dexamethasone, 3-isobutyl-1-methylxanthine (IBMX), indomethacin, insulin, and fetal bovine serum (FBS).

15. The method according to claim 11, wherein the MVs are isolated from human adipose tissue.

16. The method according to claim 11, wherein the culturing of step (a) comprises incubating the MSCs in the adipocyte differentiation medium at about 37° C. for at least about 17 days.

17. The method according to claim 11, wherein the adipocyte maintenance medium comprises RPMI, DMEM, B-27 supplement, insulin, indomethacin, and FBS.

18. The method according to claim 11, wherein the culturing of step (c) comprises incubating the MV/pre-adipocyte suspension in the adipocyte maintenance medium at about 37° C. for at least about 7 days.

19. The method according to claim 11, wherein the suspension of committed pre-adipocyte cells further comprises collagen.

20. The method according to claim 19, wherein the volume of collagen in the suspension of committed pre-adipocyte cells is about 30%.

21. A functional, vascularized adipocyte organoid produced according to the method of claim 11.

22. A method of screening a compound for pharmacological or toxicological activity, the method comprising:
(a) providing a vascularized organoid or spheroid comprising stromal cells and isolated microvessel (MV) fragments;
(b) administering a test compound to the organoid or spheroid; and
(c) detecting a pharmacological or toxicological response of the organoid or spheroid.

23. The method according to claim 22, wherein the organoid is an adipocyte organoid.

24. The method according to claim 23, wherein the response comprises one or more of cell death; cell growth; cell differentiation; change in inosculation of microvessels; change in organoid or spheroid diameter; change in organoid or spheroid size; upregulation or downregulation of production of a biomarker; and change of performance in a functional assay.

25. The method according to claim 24, wherein the response comprises upregulation or downregulation of production of a biomarker selected from the group consisting of adiponectin, PPAR-γ, GLUT4, IL-6, IL-1, and TNF-α.

26. The method according to claim 24, wherein the functional assay is selected from the group consisting of a glucose uptake assay, an insulin signaling assay, and a lipolysis assay.
